(19) United States
(12) Patent Application Publication
(10) Pub. No.: US 2006/0173522 A1
(43) Pub. Date: Aug. 3, 2006

(54) Anchoring of a medical device component adjacent a dura of the brain or spinal cord

(75) Inventor: Ivan Osorio, Leawood, KS (US)

Correspondence Address: BANNER & WITCOFF, LTD., TEN SOUTH WACKER DRIVE, SUITE 3000, CHICAGO, IL, 60606, US

(73) Assignee: Medtronic, Inc., Minneapolis, MN (US)

(21) Appl. No.: 11/339,108

(22) Filed: Jan. 25, 2006

Related U.S. Application Data
(60) Provisional application No. 60/648,628, filed on Jan. 31, 2005.

(51) Int. Cl. A61N 1/05; A61B 5/04; A61K 9/22

(52) U.S. Cl. 607/116; 604/891.1; 604/892.1; 600/378; 600/383

(57) ABSTRACT

A method and implantable medical device system capable of being anchored in a head or spinal cord of a patient. During implant, the dura of the patient is partially removed and the medical device component is placed on one side of the dura. The medical device component contains one or more anchors that mate with one or more nuts placed on the other side of the dura. The medical device component is thereby attached to the dura in a substantially fixed position relative to the head or spinal cord.

This patent application claims priority to U.S. Provisional Application Ser. No. 60/648,628 filed Jan. 31, 2005 (Attorney Reference No. 011738.00216), which is incorporated herein by reference in its entirety.

### FIELD OF THE INVENTION

The invention relates generally to implantable medical devices and more particularly to techniques for anchoring a component of a medical device system adjacent a dura membrane in a patient.

### BACKGROUND OF THE INVENTION

Nervous system disorders affect millions of people, causing death and a degradation of life. Nervous system disorders include disorders of the central nervous system, peripheral nervous system, and mental health and psychiatric disorders. Such disorders include, for example without limitation, epilepsy, Parkinson's disease, essential tremor, dystonia, chronic pain, spasticity, paralysis, sphincter dysfunction and multiple sclerosis (MS). Additionally, nervous system disorders include mental health disorders and psychiatric disorders which also affect millions of individuals and include, but are not limited to, anxiety (such as general anxiety disorder, panic disorder, phobias, post traumatic stress disorder (PTSD), and obsessive compulsive disorder (OCD)), mood disorders (such as major depression, bipolar depression, and dysthymic disorder), sleep disorders (narcolepsy), obesity, and anorexia.

As an example, epilepsy is one of the more prevalent serious neurological diseases that spans across all ages. Epilepsy generally refers to a group of neurological conditions in which a person has recurrent seizures which result from excessive neuronal discharges, which may be likened to an intermittent electrical overload, and manifests with sudden, recurrent, and transient changes of mental function, sensations, perceptions, and/or involuntary body movement. Because epileptic seizures are unpredictable, epilepsy affects a person's employability, psychosocial life, and ability to operate vehicles or power equipment. It is a disorder that occurs in all age groups, socioeconomic classes, cultures, and countries.

There are various approaches in treating nervous system disorders such as epilepsy. Treatment therapies can include any number of possible modalities alone or in combination including, for example, electrical stimulation, magnetic stimulation, drug infusion, and/or brain temperature control. Each of these treatment modalities can be operated using open- or closed-loop feedback control. For example, closed-loop feedback control techniques receive from a monitoring element a signal that carries information about a change in the state of the system, such as the brain. Such a signal can include, for example, electrical signals (such as EEG, ECoG, and/or EKG), chemical signals, other biological signals (such as change in quantity of neurotransmitters), temperature signals, pressure signals (such as blood pressure, intracranial pressure or cardiac pressure), respiration signals, heart rate signals, pH-level signals, and peripheral nerve signals (cuff electrodes on a peripheral nerve). Monitoring elements can include, for example, recording electrodes or various types of sensors.

Systems for the treatment of nervous system disorders may provide electrical stimulation or drug infusion to the brain of a patient and/or may utilize monitoring elements that sense a signal from the brain. For example, U.S. Pat. No. 5,995,868 discloses a system for the prediction, rapid detection, warning, prevention, or control of changes in activity states in the brain of a patient.

A typical electrical brain stimulation system comprises an implantable pulse generator operatively connected to the brain by a lead. The lead may serve to sense electrical activity within the brain and/or may deliver electrical stimulation to the brain. The lead has one or more electrodes at its distal end, designed to be implanted within the patient's head at a precise location, so that the electrodes are optimally and safely positioned for the desired stimulation and/or sensing. The lead is connected to the pulse generator at its proximal end. The lead body is typically anchored, with respect to a burr hole that is drilled in the patient's skull or cranium, in order to reliably and securely hold the distal end which carries the electrodes. Likewise, in the case of a catheter for providing fluid to the brain or for providing drainage, it is necessary to be able to secure the distal portion of the catheter that passes through the skull and transfers the fluid at a predetermined exact location within the brain. In such embodiments, a number of systems exist for anchoring the lead within the brain including for example, U.S. Pat. Nos. 5,86,842; 5,464,446; and 5,865,843.

In the case where the lead is a paddle-style lead (e.g., strip or grid electrode), the lead is tethered to a cable or tube and is often simply placed under the dura (a fibrous membrane that envelops the brain). The lead may only be anchored relative to a burr hole but the body itself may “float” over the cerebrospinal fluid/cortex interface or cerebrospinal fluid/spinal cord/spinal roots interface. Accordingly, if the patient moves or turns his/her head, the paddle-style lead moves with the patient and relative to the brain. As explained further below, this movement by the lead relative to the brain, however, may affect the efficacy of the nervous system disorder being treated.

As discussed, the electrodes on the paddle-style lead may be recording electrodes to sense brain electrical activity (BEA). BEA is a reliable index of brain state and function, and it allows distinction between both normal states (e.g., wakefulness, sleep and its different substates, such as NREM and REM) and abnormal states (e.g., the ictal and inter-ictal substates of an epileptic brain). BEA plays a critical role in the evaluation and treatment of nervous system disorders (e.g., pharmaco-resistant disorders, or intractable disorders, epilepsy, and movement disorders) and is an important aspect of close-loop systems for the treatment of a nervous system disorder. For example, BEA is the basis for real-time automated detection and prediction of the clinical onset of seizures. See, e.g., U.S. Pat. No. 5,995,868 issued Nov. 30, 1999 to Ivan Osorio et al.

The efficacy of such closed-loop systems, however, depends on obtaining reliable and correct BEA information. Where the paddle-style lead is susceptible to movement relative to the brain (especially during movement of the patient's head), however, the recording electrodes may not provide accurate BEA information of the desired portion of the brain.

As a result of the ever changing movement of the lead relative to the head, BEA signal quality is often poor. Signal quality depends to some extent on the amount of cerebro-spinal fluid, which acts as a shunt upon the surface of the brain, and the firmness with which the electrode rests upon the cortical surface. Signal degradation also occurs because the recording surfaces are not anchored in close contact and in a fixed position relative to the underlying cortex but, instead, “float” over the cerebro-spinal fluid. Also, since the paddle-style lead in which the recording contacts are embedded enter the cranium at an angle due to the manner in which they are tethered, the contacts closest to the point of entrance are often not in contact with the cortex, while those farthest away from the point of entrance tend to move vertically and laterally, either flapping or fluttering, thereby compromising the quality of the BEA signals. As another example, where a strip-electrode is utilized, the strip electrode is often susceptible to kinking, bending, or twisting when it is inserted through the burr hole, thereby preventing it from recording desired brain regions and potentially increasing the trauma to the cortex. Often, electrode re-insertions may be required.

As another example, the paddle-style lead may deliver electrical treatment therapy to the patient. The ability of the electrical stimulation to reach a specific target within the brain or spinal cord may directly affect the efficacy of the treatment therapy. For the reasons discussed above, because the paddle-style lead is susceptible to movement or “migration” relative to a site in the brain or spinal cord, it may greatly impact the efficacy of the treatment therapy being delivered to targeted sites within the brain due to: a) low spatial precision causing the therapy to reach the intended target only partially or not at all; b) reduced charge density due to cerebral spinal fluid (CSF) shunting or reduced drug dose due to dilution caused by the CSF.

Although the abovementioned examples relate to electrical stimulation systems, similar issues will also apply to drug delivery systems. Moreover, similar issues also apply to systems where the therapy delivery site is the spinal cord.

Thus, it would be an advancement in the art to provide a method or apparatus that can anchor a therapy component in the dura surrounding the brain or the spinal cord.

### BRIEF SUMMARY OF THE INVENTION

In an embodiment, a method and a medical device system is disclosed for anchoring a medical device component adjacent the dura of a patient. The anchored medical device component may be any component of a medical device system, including, for example, a therapy delivery element, a monitoring element to sense a neurological condition, and/or a therapy device to deliver treatment therapy to the therapy delivery element and to receive neurological signals from the monitoring element. The medical device system may be either open- or closed-loop and the same element may be used for sensing and therapy delivery.

The medical device component, endowed with mating elements in pre-specified locations (preferably along edges), is placed adjacent one side of the dura of the patient. Next, one or more mating elements are positioned adjacent the other side of the dura and fastened to the corresponding mating elements that are associated with the medical device component. The resulting assembly provides a device that is anchored to the dura by the mating elements to ensure that the medical device component is in a substantially fixed position relative to the dura and to the brain/spinal cord. Moreover, the medical device component may be either implanted epidurally or subdurally using this technique. Even further, the medical device component may be implanted radially to the dura and secured to it using mating elements. In an alternative embodiment, the medical device component may replace a portion of the dura and thereby be sutured to the remaining portions of the dura.

In one embodiment, the mating elements are an anchor and a nut, respectively. However, it will be appreciated that any mating element assembly may be used including, for example, any male/female connector assembly. The mating elements disclosed herein may be adapted to allow subsequent removability and replacement of the medical device component

Exemplary surgical tools also disclosed herein to facilitate implant of a medical device component adjacent a dura in accordance with an embodiment of the invention.

In another embodiment, either or both mating pair devices are identifiable by scan or MRI so that they can be utilized as a reference guide for points in the brain or spinal cord.

### BRIEF DESCRIPTION OF THE DRAWINGS

FIG. 1 schematically depicts an implantable medical device system that provides treatment therapy to the brain and/or monitors a characteristic of the brain.

FIG. 2 depicts a paddle-style neural lead implantable adjacent the dura in the head in accordance with the embodiment of FIG. 1, wherein the lead is a therapy delivery element or a monitoring element.

FIG. 3 depicts cross-sectional view of the mating element and the associated mating element in accordance with an embodiment of the invention.

FIGS. 4A-D depict other embodiments of mating pairs that could be implemented in accordance with the invention.

FIGS. 4E-F depict embodiments of epidural and subdural anchoring of medical device components, respectively.

FIGS. 5A-F depict embodiments of the invention wherein the medical device component implanted adjacent the dura is a catheter device for delivery of one of more chemicals such as drug.

FIG. 6 is a flow diagram depicting the overall process for anchoring the medical device component adjactent a dura of the head.

FIGS. 7A-B depict other embodiments wherein the medical device component is implanted and replaces a portion of the dura.

FIG. 8A depicts an embodiment of the invention wherein the medical device component anchored to the dura is a therapy device to deliver treatment therapy to the therapy delivery elements and/or to receive neurological signals from the monitoring elements.

FIGS. 8B-C depict an embodiment of radially implanting a medical device component and a means to secure it to the dura.

FIGS. 9-11 depict exemplary embodiments of surgical tools to facilitate implanting of a medical device component adjacent the dura.

FIG. 12 depicts an embodiment of the invention wherein one or more of the mating elements may be utilized as reference guides for localizing brain regions or structures.

### DETAILED DESCRIPTION OF THE INVENTION

The invention may be embodied in any implantable medical device system wherein a component of the system is to be implanted epidurally (between the dura and the skull) or subdurally (between the dura and the cortex) within a patient's head. In addition, the same principles may be applied to implant a medical device component either epidurally or subdurally relative to dura of the spinal cord. The medical device component may be any component of a medical device system including, for example, a therapy delivery element (electrical lead, paddle-style lead, electrode, catheter, etc.), a monitoring element to sense a neurological condition (recording electrode, sensor, etc.), and/or a therapy device (implantable pulse generator, pump, passive delivery device, etc.) to deliver treatment therapy to the therapy delivery element and to receive neurological signals from the monitoring element. As described herein, the medical device component to be implanted within the patient is anchored to the dura. The medical device component may have physical attributes (width/thickness, consistency and weight) that will avoid or minimize the potential for causing injury to the brain or spinal cord, while maximizing contact between its surfaces and the therapy targets or sites.

The invention may utilize various treatment therapies for treating nervous system disorders. Treatment therapies can include any number of possibilities alone or in combination including, for example, electrical stimulation, magnetic stimulation, drug infusion, brain temperature control (e.g., cooling), and/or providing a sensory warning to the patient/clinician.

Each of these treatment modalities may be operated using closed-loop feedback control or using open-loop therapy. Such closed-loop feedback control techniques receive one or more signals that carry information about a symptom or a condition of a nervous system disorder. Such signals can include, for example, electrical signals (such as EEG, ECoG and/or EKG), chemical signals, biological signals (such as change in quantity of neurotransmitters), temperature signals, pressure signals (such as blood pressure, intracranial pressure or cardiac pressure), respiration signals, heart rate signals, ph-level signals, and/or peripheral nerve signals (cuff electrodes on a peripheral nerve). Such signals may be recorded using one or more monitoring elements such as monitoring electrodes or sensors. For example, U.S. Pat. No. 6,227,203, assigned to Medtronic, Inc., provides examples of various types of monitoring elements that may be used to detect a symptom or a condition or a nervous system disorder and responsively generate a neurological signal.

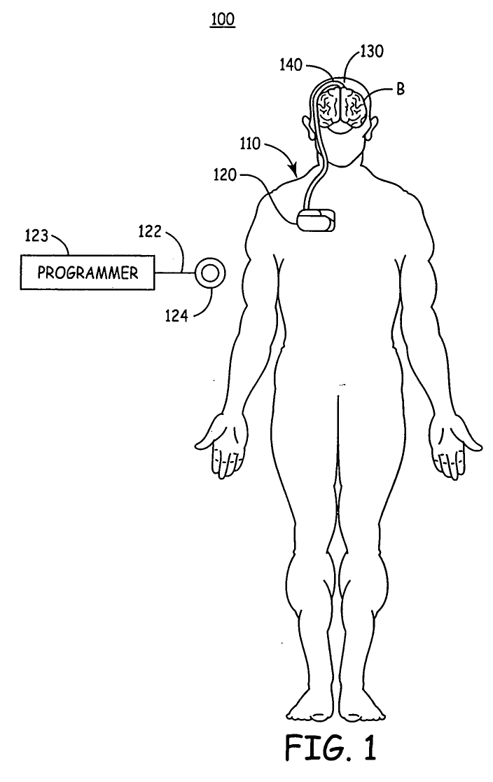

In accordance with one set of embodiments, the medical device component to be implanted within the head is a therapy delivery element or a monitoring element or a combination of both (dual function). FIG. 1 schematically represents these embodiments and depicts an implantable medical device system 100 that provides treatment therapy to the brain and/or monitors a characteristic of the brain B.

The medical device system 100 generally includes a device 120 capable of being implanted in a patient 110 and coupled to one or more anchored therapy delivery elements 130 and/or one or more anchored monitoring elements 140. The therapy delivery elements 130 deliver treatment therapy to the neurological tissue in the patient (e.g., the brain). Likewise, the monitoring elements 140 monitor one or more characteristics of the neurological tissue (e.g., brain, spinal cord or other organ) and can be the same device as the therapy delivery elements 130. The implantable device 120 may continuously or intermittently communicate with an external programmer 123 (e.g., patient or physician programmer) via telemetry using, for example, radio-frequency signals and having a coil 124 and a lead 122 coupling the programmer 123 with the coil 124. The external programmer 123 may be any general-purpose computing device (e.g., personal computer, hand-held device, etc.) having an operating system configured with custom external system application software. Other communication techniques, of course, may also be utilized including a telemetry channel.

In the embodiment where the medical device system 100 is brain stimulation system, the medical device system 100 delivers electrical stimulation to the brain through the therapy delivery elements 130. In the event that the medical device system 100 also utilize closed-loop feedback control, the medical device system 100 monitors BEA or some other signal from the monitoring elements 140, conditions the brain signals for processing, determines the onset, presence, and/or intensity of any neurological event, configures the parameters for delivering electrical stimulation through the therapy delivery elements 130 if any should be provided.

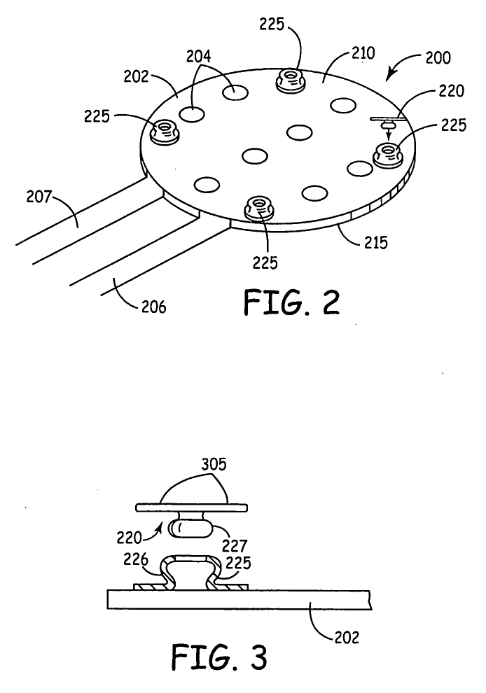

FIG. 2 depicts a paddle-style neural lead 200 and is in accordance with the embodiment of FIG. 1 wherein the medical device component to be implanted within the head is the lead 200 having one or more therapy delivery elements 130 and/or one or more monitoring elements 140. Paddle-style leads are generally known in the art and, therefore, can take any shape or form or surface dimension including, for example, those paddle-style leads depicted in U.S. Pat. No. 6,038,480. As defined herein, paddle-style leads may also include strip-electrodes and grid-electrodes. In accordance with the invention, however, the paddle-style neural lead 200 of FIG. 2 has associated mating elements 225 (which may be on either side, as discussed herein, depending on their placement in reference to the side that contains the electrodes or to the epidural or subdural sides). The type (male or female) of the mating element 225 may depend on the epidural or subdural placement.

Neural lead 200 includes a body 202, a plurality of electrodes 204 (which may be recording and/or stimulating) and a conduit 206 connected to the body 202 for carrying electrical signals or therapeutic substances. The conduit 206 may be placed orthogonal to the surface of the body 202, rather than parallel to it, as it is customary, to avoid uneven distribution of forces on the body 202, dura and cortex. Conduit 206 exits through a surgical opening in the skull. Optionally, neural lead may also include a second conduit 207 for carrying a separate channel of conducting wires or tubes. The neural lead 200 also includes a plurality of mating elements 225, and particularly in this embodiment, the mating elements 225 are anchor shafts. The mating elements 225 are disposed on a side 210 of the body 202, which is the side to be placed adjacent to the dura membrane; the mating elements 225 may be placed anywhere on the surface to ensure lead stability in reference to and in contact with the target. The mating elements 225 are paired with associated mating elements 220, and particularly in this embodiment, the associated mating elements 220 are nuts. The mating element 225 and associated mating element 220 attachable relative to each other and may be positioned such that they “sandwich” the dura membrane in between.

The body 202 in FIG. 2 is for placement on the side of the dura facing the bone (“epidural”); the electrodes or sensors 204 and the mating elements 225 face down in the direction of the cortex. In the embodiment where the body 202 for placement on the side of dura is facing the cortex (“subdural”), mating elements 225 would be facing in the direction of the bone and the electrodes or sensors 204 may be facing in the direction of the sensor (although in some applications, it may be desirable to have the electrodes face the skull). Surgical openings in the dura for the mating elements 225 may be lined with a ring made of a biocompatible material to avoid tearing.

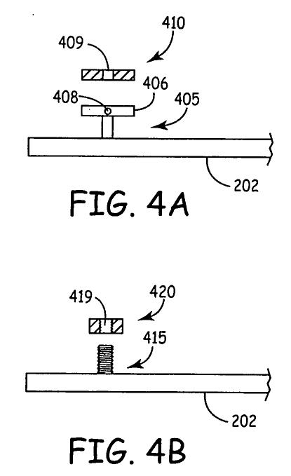

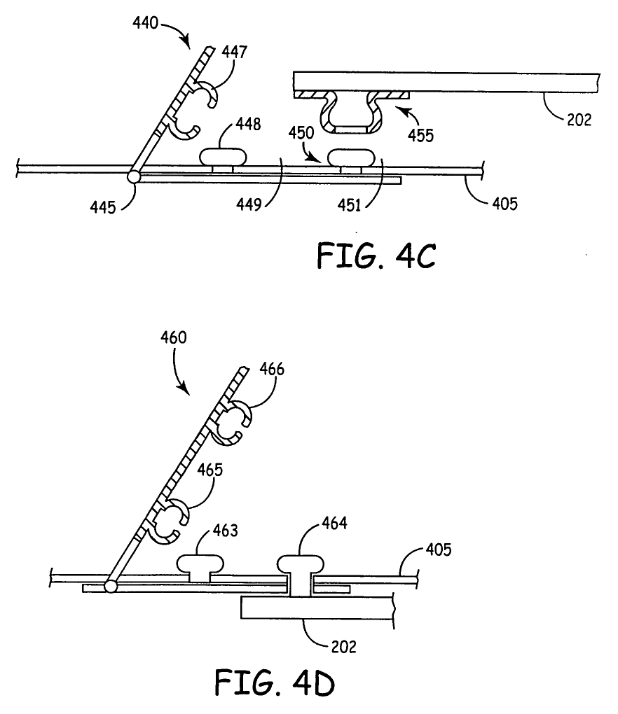

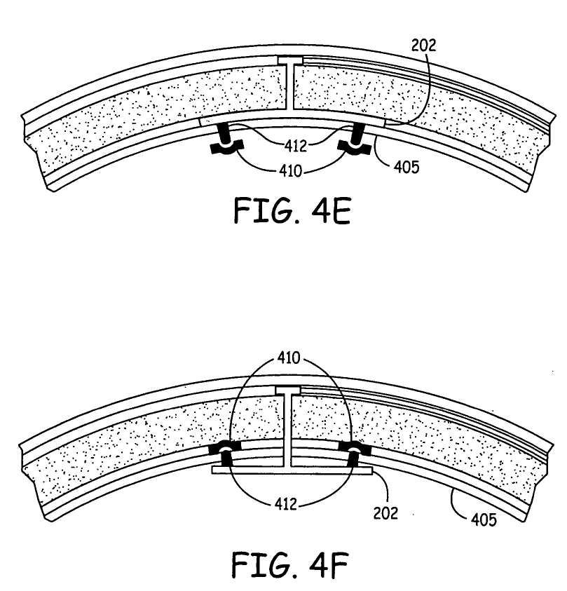

As discussed herein, neural lead 200 may be placed epidurally (on the side facing the bone) or subdurally (on the side facing the cortex). For example, in the treatment of pain, the lead 200 is likely to be used epidurally. In the case of epilepsy, it is likely that the neural lead 200 would be used subdurally. See FIGS. 4E and 4F. In either case, the appropriate surgical procedure for optimal placement is performed, as discussed herein, and the body 202 is positioned beneath the cranium or vertebral structures.

The number, configuration (shape and size) and position of the electrodes 204 may vary greatly within the scope of the invention. The shape and dimensions of the body 202 may also vary greatly and still be considered within the scope of the invention. Moreover, the electrodes 204 may take the form of any type of the monitoring elements and/or therapy delivery elements discussed herein.

FIG. 3 depicts cross-sectional view of the mating element 225 and the associated mating element 220. As depicted, the mating element 225 has a curved wall 226. Similarly, the associated mating element 220 has a corresponding curved wall 227. For epidural or subdural placement, the two components may be “snapped” together with the dura membrane placed in between and fixed by way of flanges 305 on the associated mating element 220. For subdural implant, associated mating element 220 may be initially attached to body 202 to enable temporary stabilization of the body 202. In one embodiment, piercing of the dura may be achieved by the associated mating element 220, that in the distal-most part may be shaped like an stylet. The stylet portion may have a removable protective cap (not shown) to prevent accidental piercing of the brain.

Again, the anchor and nut devices described above are merely exemplary embodiments and any number of mating pair devices may be used. For example, any male/female mating pair devices may be used such as a threaded screw and a nut. As other examples, shafts, pins, screws, magnetic devices, or semi-adhesive devices may also be used. Moreover, either mating pair may be associated with the medical device component. The mating elements may be made of any type of material including, for example, platinum, titanium, or plastic. In one embodiment as discussed herein, the mating element is detectable by brain scanning such as an MRI.

FIGS. 4A-D depict other embodiments of mating pairs that could be implemented in accordance with the invention. FIG. 4A depicts a “cuff-link” type mating pair wherein the mating element 405 has a member 406 that is rotatable relative to an axis 408. The member 406 is rotated to a vertical position to insert the member through an aperture 409 of the associated mating element. Once through, the member 406 may be rotated back to its horizontal position. FIG. 4B depicts a screw and bolt mating pair wherein the mating element 415 is a “screw-like” device having threads. The associated mating element 420 has corresponding threads in the aperture 419 that can receive the mating element 415. In an embodiment, the mating pairs may detachable in the event that the medical device component is subsequently replaced.

FIG. 4C, in particular, permits subsequent detachability of the medical device component 202 while maintaining designated anchors in the dura 405 to act as fiducials for precise repositioning of the device. In this embodiment, a plurality of connectors 440 may be provided wherein each connector 440 is attachable to the dura 405. Each connector 440 has a hing 445 that is adapted to attach and/or “sandwich” the dura 405 between a pair of mating elements 447 and 448. The pair of mating elements 447 and 448 may be any mating pair including an anchor and nut as illustrated. The connector 440 also has a third mating element 450 that mates with an associated mating element 455 that is part of the medical device component, in this case the paddle 202. As apparent in these embodiments, the medical device component may thereby be subsequently removed while leaving the plurality of connectors 440 in fixed positions relative to the dura 405 and brain or spinal cord. In an embodiment, where the dura is replaced by the medical device component, the medical device component may be positioned at reference points 459 or 451 of FIG. 4C.

FIG. 4D depicts another orientation similar to that of FIG. 4C except where the medical device component 202 is subdurally implanted. The connector 460 is similar in structure of connector 440 of FIG. 4C. Mating element 463 is associated with connector 440 while mating element 464 is associated with medical device component 202. As disclosed, mating element 464 is positionable between apertures in connector 460 and the dura 405 and is matable with a mating element 466 that is associated with connector 460. Similarly, mating element 463 is positionable between a second aperture in the dura 405 and is matable with mating element 465 that is associated with connector 460. Mating element 466 may be removable.

As discussed, the medical device component 202 may be implanted either epidurally or subdurally. FIG. 4E depicts an embodiment wherein the medical device component 202 is epidurally implanted adjacent the dura 405, wherein each pair of mating element 410 and associated mating element 412 “sandwiches” the dura, thereby anchoring the medical device component in a fixed position relative to the dura and the brain. Similarly, FIG. 4F depicts an embodiment wherein the medical device component 202 is subdurally implanted adjacent the dura 405. Again, as discussed, although the embodiments disclosed herein are with reference to the brain, the same principles may be applied for anchoring a medical device component to dura associated with a spinal cord of a patient.

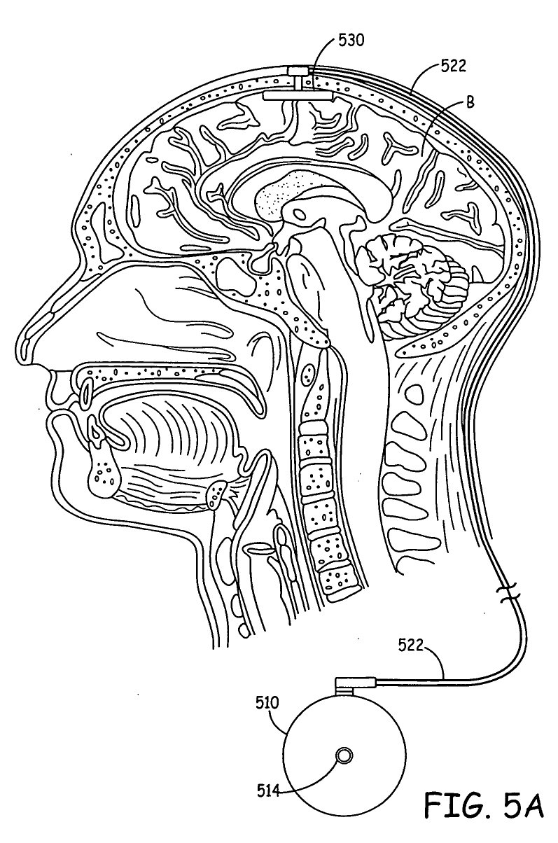

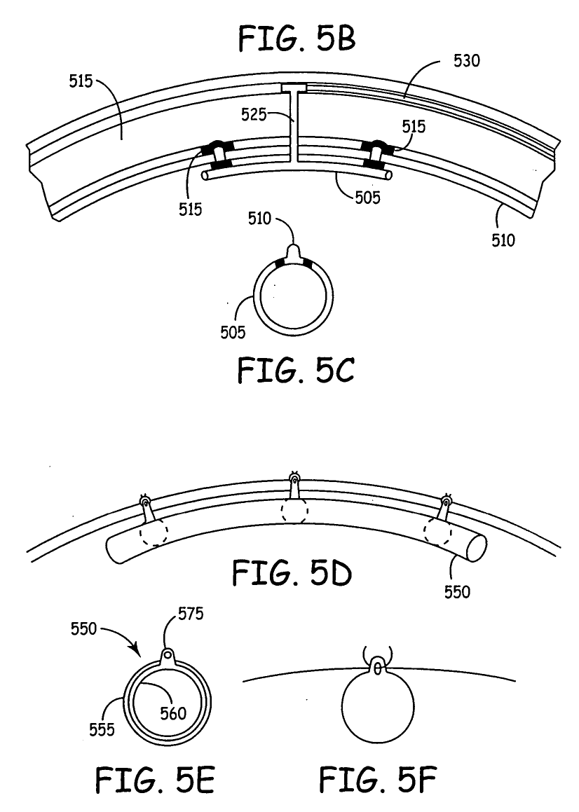

Again, the medical device component may also be a catheter device for delivery of one or more chemicals such as drug. As illustrated in FIG. 5A, the catheter 530 may be implanted adjacent the dura as disclosed herein and may be coupled to an infusion pump 510 or a passive release device (not shown). See, e.g., U.S. Pat. No. 4,692,147. The pump 510 or passive release device may be implanted below the skin or scalp of a patient for delivery of drug (or any other chemical) as the form of treatment therapy for a patient. The pump 510 or passive release device has a port 514 into which a needle can be inserted through the skin to inject a quantity of a agent, such as a medication or drug. The agent is delivered from device 510 through a catheter port 522 into the catheter 530. The catheter 530 is positioned to deliver the agent to specific infusion sites in the brain.

FIGS. 5B-F depict various embodiments for anchoring a catheter adjacent the dura. Referring to FIGS. 5B and 5C, the catheter 505 may have a perimeter that is of a geometry and material that is capable of being attached to the dura 510 (e.g., attached using mating elements similar to that illustrated in FIG. 4 along with sutures). Optionally, the catheter 505 has a member 525 that exits through the skull 515 and to a conductor 530 to the medical device system. Member 525 may be of a flexible structure and material so as to minimize any potential stress on the catheter 505. Catheter 505 has an a mating element 510 that may be passed through a slit or aperture in the dura 505 and attached to an associated mating element 515 to thereby anchor the catheter 505 to the dura 510. The procedure for attaching such a device is similar to that described above and the mating element pairs can be of any number of forms including those disclosed above.

FIGS. 5D-F depicts another embodiment for anchoring a catheter 550. Catheter has an outer wall 555, an inner wall 560, and one or more mating elements 575 formed partially within the catheter walls 555 and 560. Second one is with the female being a hole and can use a wire or suture to tie it to the dura.

Alternatively, the medical device component may simply be an electrode itself. Such an electrode may be different than a traditional lead (i.e. wireless coupling to a medical device) and/or it may just be a part of the mating components that attach to the dura.

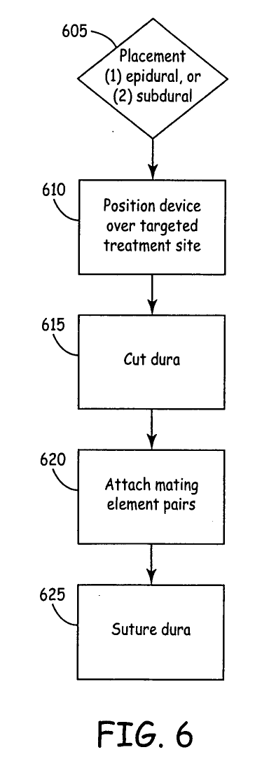

FIG. 6 is a flow diagram depicting the overall process for anchoring the medical device component of FIG. 1 to the dura. At step 605, the physician chooses between epidural and subdural placement of the medical device component. At step 610, assuming epidural placement is chosen, the medical device component is positioned such that it lies over potential/definite monitoring or therapeutic targets. At step 615, and whenever indicated, the dura membrane is incised and/or reflected to provide access to its inferior surface or subdural side of the dura. Depending on the geometry of the medical device component to be implanted and the structure of mating pair devices being used, the type and extent of the incision in the dura may vary. For example, a simple incision may be made or, alternatively, the dura may be cut such that it may be folded back away from the brain. Alternatively, a section of the dura may be completely removed to be replaced by a lead with embedded electrodes or by catheters. The size and shape of the dura that is removed should closely match that of the lead or catheter to prevent CSF leakage into the epidural space.

At step 620, the associated mating components (or anchors) are inserted under the dura and attached or fastened to the mating components associated with the medical device component. For example, the anchors associated with the medical device component pierce the dura membrane and attach to the mating nuts on the other side of the dura. Depending on the structure of the mating elements, this may be achieved by fastening, pushing, or screwing the mating elements together. Also depending on the structure of the mating elements, the dura may not require piercing (e.g., where magnetic mating elements are used or mating elements that frictionally engage the dura). Special tools may be used to facilitate the piercing and mating process as illustrated below. The medical device component is now anchored to the dura membrane. At step 625, the dura may then be sutured back or to the medical device component that replaces it using known techniques.

The medical device component may then be coupled via leads to the other components of the medical device system such as an implantable pulse generator. Such leads would exit through a burr hole in the skull under techniques known in the art. See, e.g., U.S. Pat. No. 5,464,446. Again, as discussed, the anchor and nut devices described in the above process description are merely exemplary embodiments and any number of mating pair devices may be used.

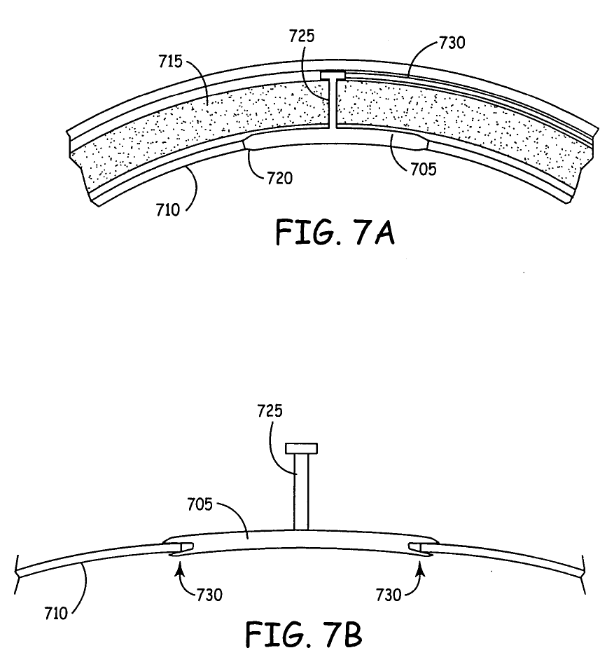

In another embodiment, the medical device component need not be placed above or underneath the dura but, instead, may replace a portion of the dura. FIG. 7A depicts a lead device 705 that is implantable in such a manner. As illustrated, the lead device 705 may have a perimeter that is of a geometry and material that is capable of being attached to the dura 710 (e.g., attached using mating elements similar to that illustrated in FIG. 4C along with sutures). Optionally, the lead device 705 has a member 725 that exits through the skull 715 and to a conductor 730 to the medical device system. Member 725 may be of a flexible structure and material so as to minimize any potential stress on the lead device 705. The perimeter 720 of the lead device 705, however, may be attached directly to the dura membrane 710 below the skull 715. The procedure for attaching such a device is similar to that described above, except that a portion of the dura 710 resembling the dimensions and shape of the medical device component 705 is permanently removed from the head and replaced by the medical device component 705. The perimeter 720 of the device 705 has a membrane that may be sutured or attached to the dura 710 in the head, thereby completely closing the opening created by the cutting of the dura 710. The membrane may be of any suturing material such as silk and may also have additional clipping elements to facilitate attachment of the membrane to the dura. In addition, the membrane may have filaments or clip elements that are radio-opaque for mapping purposes (discussed further herein). Alternatively, the membrane itself or the sutures may also be radio-opaque.

FIG. 7B depicts yet another embodiment of the medical device component 705 being anchored by replacement of the dura. In particular, the dura 710 may be positioned within leaflets 730 associated with the medical device component 705. The dura 710 may thereby be sutured or anchored using techniques discussed above.

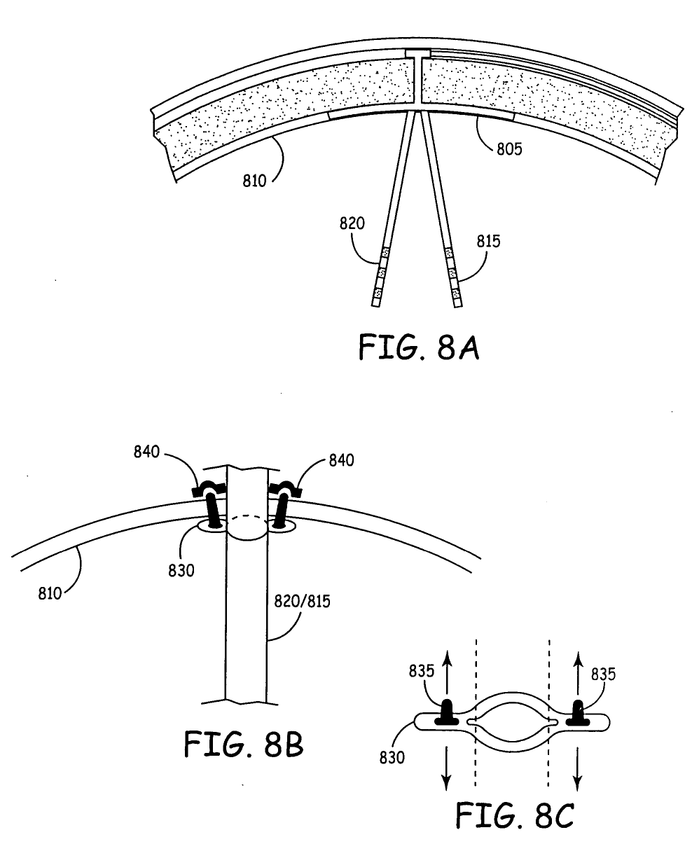

As discussed, the medical device component to be implanted adjacent the dura can be any component of a medical device system. The above embodiments illustrate the medical device component as a monitoring element and/or a therapy delivery element. The medical device component, however, can also be a therapy device such as, for example, an implantable pulse generator, a pump, or a passive release device for a compound. In such an embodiment, as illustrated in FIG. 8A, the therapy device 805 may be implanted adjacent the dura 810 and may be coupled to monitoring elements 815 and/or therapy delivery elements 820 implanted within the brain. For example, the therapy device 805 may be an implantable pulse generator that is coupled to stimulating and recording electrodes implanted within the brain. Alternatively, the therapy device 805 may be a pump or a passive release device that is coupled to catheters and/or monitoring elements implanted within the brain. The therapy device 805 may be implanted subdurally or epidurally (as shown) using techniques similar to those disclosed above. The geometry of the therapy device 805 may be configured (width/thickness, consistency and weight) to avoid or minimize the potential for causing injury to the brain while maximizing contact with it and stability in reference to the therapeutic targets and to facilitate placement of the device within the head. Again, similar techniques as those describe above may be utilized to implant a therapy device adjacent the dura.

Monitoring elements 815 and/or therapy delivery elements 820 implanted within the brain may also be radially anchored to the dura. FIGS. 8B & 8C illustrate an embodiment of radial implant of a medical device component such as a monitoring element 820 or a therapy delivery element 815. The medical device component has one or more attached fasteners 830. Associated with each fastener 830 are one or more mating elements 835 that can take the form of any of the mating elements discussed above. In accordance with the above description, the mating elements 835 may be fastened to one or more associated mating elements 840 to thereby “sandwich” the dura 810 therebetween. In an embodiment, the fasteners 830 may be slideable during implant relative to the medical device component 815/820 so as to adjust the implant depth of the medical device component 815/820. Once implanted or positioned, however, the fastener 835 may be fixed in position relative to the medical device component 815/820 (using, for example, pins, tightening, etc.) so as to avoid subsequent radial migration of the medical device component 815/820.

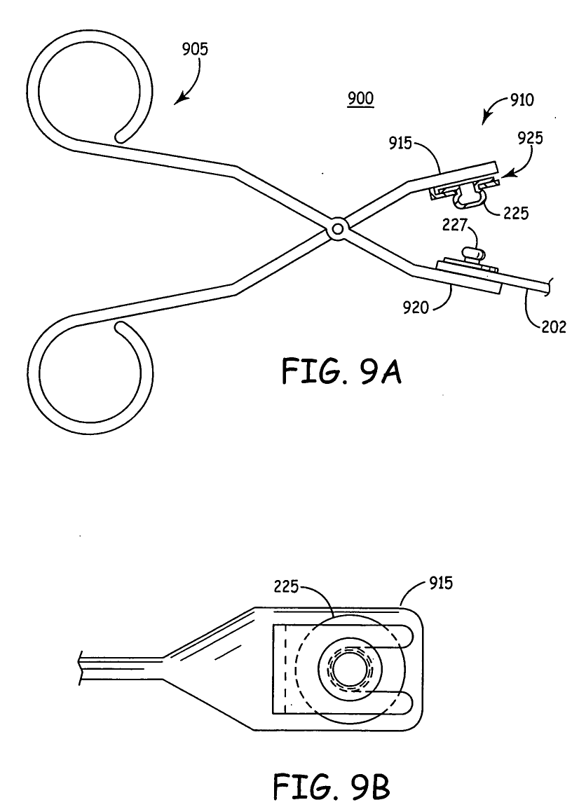

FIGS. 9-11 depict exemplary embodiments of a surgical tool that may be used to facilitate the implant or attachment of a medical device component adjacent the dura. In the embodiment of FIGS. 9A-B, the surgical tool 900 has a distal end 910 having a first prong 915 adapted to secure a first mating element 225 and a second prong adapted to hold a medical device component 202 having an associated second mating element 227. The surgical tool 900 also has a proximal end 905 to be held by a user (i.e., the surgeon) to control movement of the first and second prongs 915 and 920, wherein the proximal end 905 may close the first and second prongs 915 and 920 to mate together the first and second mating elements 225 and 227. In particular, the first prong 915 has an insert portion 925 that is capable of accepting and securing a base portion of a nut 225 within the insert portion 925. When the mating pairs are to be “snapped” together as discussed above, the medical device component 202 may be positioned between the prongs 915 and 920 of the surgical tool 900 and the surgical too may be used to fasten (i.e., force or otherwise push) the mating parts 225 and 227 together. FIG. 9B depicts a bottom view of upper prong 915 with the nut 225 positioned within the insert portion 925.

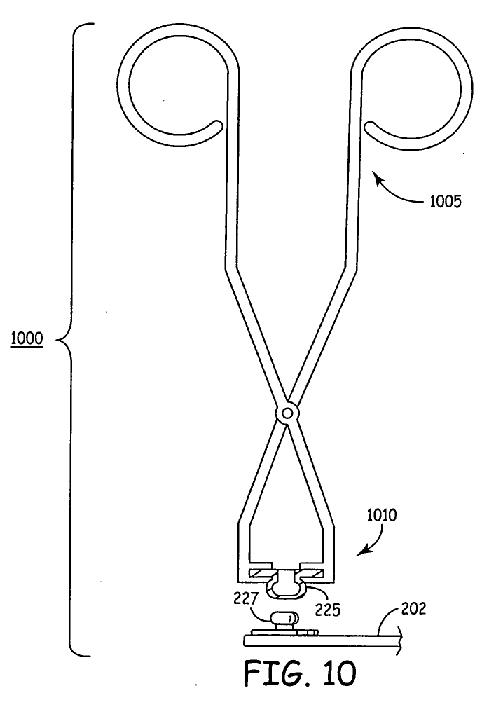

Similarly, FIG. 10 depicts a surgical tool 1000 having a proximal end 1005 and a distal end 1010 adapted to accept a mating element 225. The distal end 1010 has a first prong and a second prong adapted to secure a mating element 225. The proximal end 1005 is adapted to be held by a user to control movement of the first and second prongs, wherein the proximal end may close the first and second prongs to pry open the (female) mating element 225 for attachment to the corresponding (male) mating element 227. In this embodiment, the surgical tool 1000 may be used to effectively “pry open” the mating element 225 which once released clamps on the corresponding mating element 227 associated with the medical device component 202.

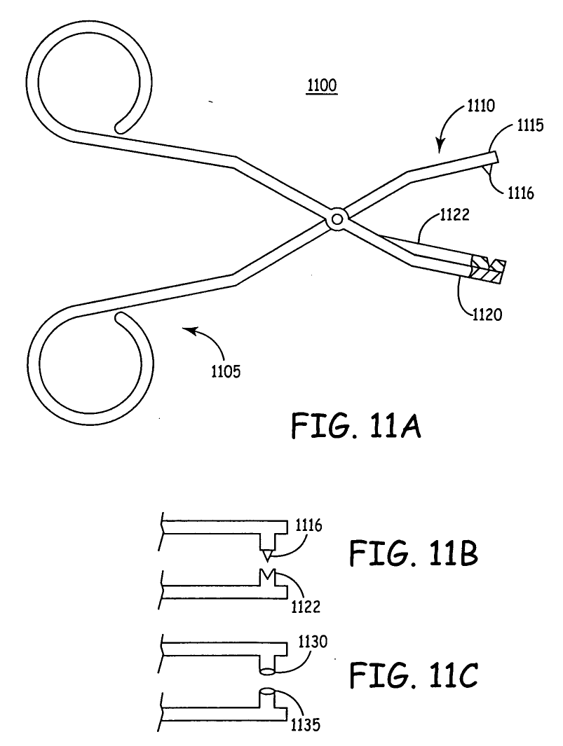

FIG. 11A depicts an exemplary embodiment of a surgical device 1100 for piercing the dura to allow snug fitting of the male element (the diameter of the piercing element at its widest is a few thousands of an inch larger than the diameter of the male element). The surgical device has a proximal end 1105 and a distal end 1110 having a pair of prongs 1115 and 1120. The proximal end 1105 is adapted to be held by a user to control movement of the first and second prongs 1115 and 1120. One prong 1115 has a piercing element 1116 and the other prong has 1120 support element 1122, to thereby accept interconnection of a first and second mating element surrounding the dura. See FIG. 11B depicting the piercing element 1116 and the support element 1122. The dura may be place between the prongs 1115 and 1120 and may be pierced by forcing down the piercing elements 1116 over the dura. Alternatively, as shown in FIG. 11B, the piercing element 1130 and the support element 1135 may be of a structure to actually cut the dura to create an aperture. In the embodiment, the piercing element and the support element are mating cylindrical components to effectively “hole punch” the dura.

In an embodiment, either or both mating pair devices are identifiable in imaging studies such as an MRI. As such, to allow for imaging, the mating element may be made of a radio-opaque or like material. Advantageously, the mating pair devices, such as nuts, can be utilized as a reference guide for localizing brain regions or structures. In the event that a subsequent invasive procedure is required to replace or reinsert the device or to put in a new medical device component within the head, preserving the same spatial relationship between it and the monitoring and/or therapeutic target, the mating devices such as those in FIG. 9C may be left in place. In the case of invasive evaluation for intractable epilepsy (where the device has been used for localization of epileptogenic tissue and for cortical mapping and the actual resection (topectomy) of abnormal tissue is deferred (in which case the medical device may be removed to allow full closure of the dura and scalp thus decreasing risk of infection) the mating devices are left in place to serve as fiducials or markers to allow precise localization of the surgical targets at a later date. In case the medical device is left in place, the mating pairs anchor it, preventing movement or migration so that if more surgery is required at a later date, the spatial relation between the device and the monitoring or therapeutic target is fully preserved.

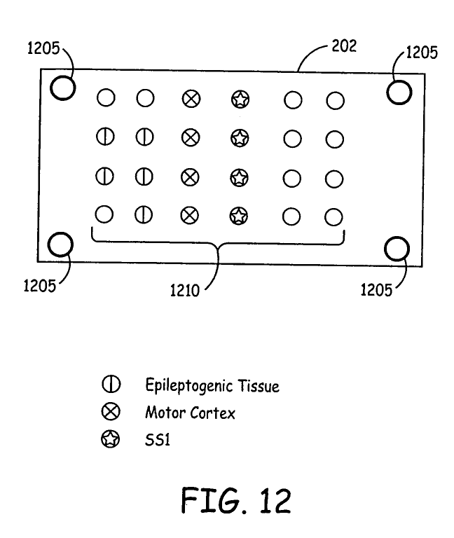

Use of mating elements as reference guides for imaging is illustrated with reference to FIG. 12. FIG. 12 depicts a medical device component 202, in this case a paddle-style electrical lead, implanted adjacent a dura of a patient and having four pairs of mating element pairs 1205 and an array of electrodes 1210. Through known stimulation and recording techniques (e.g., evoking potentials and recording responses), various portions of the brain may be mapped with reference to the mating element pairs. For example, as illustrated in FIG. 12, certain electrodes may signify regions of epileptogenic tissue, the motor cortex, and the somatosensory cortex (SS1). These regions of the brain may thereby be mapped and subsequently identified with reference to one or more of the mating element pairs. Once mapped, the medical device component 202 and extraneous mating element pairs may even be removed. In case surgery is deferred, or even if it is not since the medical device needs to be removed to perform the resection, the mating elements may be used to precisely localize (via direct visual inspection and measurements) the areas to be resected, while avoiding those that should not be resected. Also, the mating elements may be used to, at a later time, localize without need for any procedures other than imaging, such as MRI, various regions of the brain such as for example, the motor cortex, the sensory cortex, the visual cortez, the auditory cortex, and language areas (receptive and expressive) or track the growth of a lesion. Mating elements may be used exclusively as fiducials for localization of cortical regions or deep brain structures.

Those skilled in the art will appreciate that the medical device systems described above may take any number of forms from being fully implanted to being mostly external and can provide treatment therapy in any number of forms. For example, the treatment therapy being provided by the medical device systems may vary and can include, for example, electrical stimulation, magnetic stimulation, drug infusion, and/or brain temperature control (e.g., cooling). Moreover, as mentioned above, the above concepts may be utilized for spinal cord stimulation or drug delivery systems. Finally, it will be appreciated that the medical device systems may be utilized to analyze and treat any number of nervous system disorders.

Thus, embodiments of ANCHORING OF A MEDICAL DEVICE COMPONENT ADJACENT A DURA OF THE BRAIN OR SPINAL CORD are disclosed. One skilled in the art will appreciate that the invention can be practiced with embodiments other than those disclosed. The disclosed embodiments are presented for purposes of illustration and not limitation, and the invention is limited only by the claims that follow.

What is claimed is:

1. A method of anchoring a medical device component in a patient comprising:
(a) placing the medical device component adjacent a first side of a dura of the patient, wherein the device component has at least one first mating element;
(b) placing at least one second mating element on a second side of the dura; and
(c) fastening the at least one first mating element with the second mating element, wherein the dura is situated between the at least one first and second mating elements to thereby anchor the medical device component to the dura.

2. The method of claim 1 wherein the medical device component is an electrical lead.

3. The medical device system of claim 1 wherein the medical device component is an electrode.

4. The method of claim 1 wherein the medical device component is an implantable pulse generator.

5. The method of claim 1 wherein the medical device component is a catheter.

6. The method of claim 1 wherein the medical device component is an implantable pump.

7. The method of claim 1 wherein the medical device component is a passive release device.

8. The method of claim 1 wherein placing the at least one second mating element comprises cutting a portion of the dura to facilitate subdural implant of the at least one second mating element.

9. The method of claim 8 wherein placing the medical device component comprises epidurally implanting the medical device component.

10. The method of claim 8 further comprising replacing the removed portion of the dura.

11. The method of claim 1 wherein the first side of the dura is an epidural side and the second side of the dura is a subdural side.

12. The method of claim 1 wherein the first side of the dura is a subdural side and the second side of the dura is an epidural side.

13. The method of claim 1 wherein the at least one first mating element is a male connector and the at least one second mating element is a female connector.

14. The method of claim 1 wherein the at least one first mating element is a female connector and the at least one second mating element is a male connector.

15. The method of claim 1 wherein at least one of the mating elements is adapted to be detectable by imaging.

16. A medical device system comprising:
(a) a medical device component adapted to be attached to a dura of a patient;
(b) at least one first mating element associated with a side of the medical device component; and
(c) at least one second mating element adapted to being fastened to the at least one first mating element, wherein the dura is situated between the at least one first and second mating elements to thereby anchor the medical device component to the dura.

17. The medical device system of claim 16 wherein the medical device component is an electrical lead.

18. The medical device system of claim 16 wherein the medical device component is an electrode.

19. The medical device system of claim 16 wherein the medical device component is an implantable pulse generator.

20. The medical device system of claim 16 wherein the medical device component is a catheter.

21. The medical device system of claim 16 wherein the medical device component is an implantable pump.

22. The medical device system of claim 16 wherein the medical device component is a passive release device.

23. The medical device system of claim 16 wherein the medical device component is adapted to be subdurally implanted.

24. The medical device system of claim 23 wherein the at least one second mating element is adapted to be epidurally implanted.

25. The medical device system of claim 16 wherein the medical device component is adapted to be epidurally implanted.

26. The medical device system of claim 25 wherein the at least one second mating element is adapted to be subdurally implanted.

27. The medical device system of claim 16 wherein the at least one first mating element is a male connector and the at least one second mating element is a female connector.

28. The medical device system of claim 16 wherein the at least one first mating element is a female connector and the at least one second mating element is a male connector.

29. The medical device system of claim 16 wherein the at least one first mating element is selected from the group consisting of an anchor, a nut, and a screw.

30. The medical device system of claim 16 wherein the at least one second mating element is selected from the group consisting of an anchor, a nut, and a screw.

31. The medical device system of claim 16, wherein at least one mating element is detectable through imaging.

32. The medical device system of claim 16 further comprising a lead having a distal end coupled to the medical device component.

33. The medical device system of claim 32 further comprising an implantable pulse generator coupled to a proximal end of the lead.

34. The medical device system of claim 16 further comprising a catheter having a distal end coupled to the medical device component.

35. The medical device system of claim 34 further comprising an implantable pump coupled to a proximal end of the catheter.

36. The medical device system of claim 16 wherein at least one of the mating elements is adapted to be detectable through imaging.

37. The medical device system of claim 16 wherein the at least one first and second mating elements are adapted to be detachable relative to each other.

38. An apparatus for attaching a medical device component to a dura comprising:
(a) a body having a first portion and a second portion;
(b) first and second mating elements along the first portion of the body and adapted to attach to a dura; and
(c) a third mating element along the second portion of the body and adapted to attach to an associated mating element attached to a medical device component.

39. A method of anchoring a medical device component in a patient comprising:
(a) removing a portion of a dura of the patient forming an exposed portion, wherein the exposed portion has a dura perimeter;
(b) placing the medical device component adjacent the exposed portion, wherein the medical device component has a device perimeter; and
(c) attaching the device perimeter of the medical device component to the dura perimeter to thereby substantially close the exposed portion.

40. The method of anchoring of claims 39, wherein (c) comprises using mating element pairs to attach the perimeter of the medical device component

41. The method of anchoring of claims 40, wherein each mating element pair comprises a first mating element attached to the medical device component and a second mating element and (c) comprises attaching the second mating element to the dura.

42. The method of anchoring of claims 41, wherein the mating element pairs enable precise special replacement of the medical device component.

43. The method of anchoring of claims 41, wherein the mating element pairs enable precise special reinsertion of the medical device component.

44. The method of anchoring of claims 41, wherein the mating element pairs serve a reference points for localization of epileptogenic tissue.

45. The method of anchoring of claims 41, wherein at least one mating element pair serves as at least one landmark for identifying cortical regions.

46. The method of anchoring of claims 41, wherein at least one mating element pair is of a radio-opaque material.

47. A tool for attaching a medical device component to a dura comprising:
(a) a distal end having a first prong adapted to secure a first mating element and a second prong adapted to hold a medical device component having an associated second mating element; and
(b) a proximal end adapted to be held by a user to control movement of the first and second prongs, wherein the proximal end may close the first and second prongs to mate together the first and second mating elements.

48. A tool for attaching a medical device component to a dura comprising:
(a) a distal end having a first prong and a second prong adapted to secure a female mating element; and
(b) a proximal end adapted to be held by a user to control movement of the first and second prongs, wherein the proximal end may close the first and second prongs to pry open the female mating element for attachment to a male mating element.

49. A tool for attaching a medical device component to a dura comprising:
(a) a distal end having a first prong and a second prong;
(b) a proximal end adapted to be held by a user to control movement of the first and second prongs; and
(c) means on the first and second prongs for piercing the dura to accept interconnection of a first and second mating element surrounding the dura.
